(19) United States
(12) Patent Application Publication
(10) Pub. No.: US 2010/0100025 A1
(43) Pub. Date: Apr. 22, 2010

(54) TOURNIQUET DRESSING SLEEVE FOR DIGITS

(76) Inventor: RONALD KANE, CEDAR FALLS, IA (US)

Correspondence Address: MCKEE, VOORHEES & SEASE, P.L.C., 801 GRAND AVENUE, SUITE 3200, DES MOINES, IA, 50309-2721, US

(21) Appl. No.: 12/253,350

(22) Filed: Oct. 17, 2008

(51) Int. Cl. A61F 13/00; A61B 17/00

(52) U.S. Cl. 602/79; 606/203

(57) ABSTRACT

A tourniquet dressing sleeve for digits is made from a sleeve preferably non-Latex polymeric stretchable material layered with a covering layer of stretchable woven fabric then folded over upon itself to form a sleeve with the edges sewn together to form a flexible, stretchable compression sleeve to fit around digits to exsanguinate them.

### FIELD OF THE INVENTION

The field of this invention relates to tourniquet dressing sleeves to allow extra compression of digits after surgery to push their blood supply proximally to reduce swelling.

### BACKGROUND OF THE INVENTION

After surgery on digits, that is fingers and toes, the surgeon often applies a compression wrap around the digit in order to both protect the appendage and compress it. The compression wrap is typically left on for 24 hours and then removed. At this point in time (after 24 hours) the compression wrap has performed its function for both protection and swelling prevention.

Devices such as common tourniquets are frequently used in minor surgical procedures involving the digits (or toes) such as laceration repair, matrixectomies, etc. These procedures typically require a sterile field. Many surgeons, since the procedures are minor, ignore this sterile requirement and commonly use rubber bands for the tourniquet purpose.

Rubber bands however are not satisfactory for obvious reasons. As a result, products have appeared on the market such a Tourni-Cot®, a product which is a ring of silicone rubber used by applying it to a digit to stop the blood from flowing. As the ring is rolled onto the digit it exsanguinates the blood from the digit making it an exsanguinating tourniquet. This product is commonly used by hand surgeons, podiatrists and in the emergency room. They are typically individually packaged and sterile and made from silicone rubber. They offer obvious advantages because of their sleeve-like capacity in comparison with simple rubber bands. They are usually available packed 20 units per box in small, medium, large and extra large sizes.

Yet another current product is simply a wide band of latex, available from WFMED. This is nothing more than a wide latex rubber band and is available in various widths for use by surgeons as tourniquets.

There is also a tubular latex tourniquet by MEDIpoint, Inc which is simply a latex sleeve with good elasticity. The digit is simply inserted in the sleeve by rolling it upon the appendage, then it is cut to length resulting in a compressed, stretchable, sleeve.

While each of the above devices are commonly used by surgeons, each has its own inherent disadvantages. For example, a silicone band is easy to leave in place and risks potentially covering it over with dressing. The rubber band tourniquet is difficult to apply and often does not exsanguinate the digit it goes on. It can therefore be seen that there is a continuing need for further refinements and development in exsanguinating tourniquets. This invention has as its primary objective the fulfillment of this need.

Another objective is to provide an exsanguinating tourniquet which is easier to apply than those currently available, and which will function affectively as a compression wrap after it functions to exsanguinate blood from the digit or toe. Finally, it is also an objective to provide one which is more attractive as a bandage for the patient.

The method and means of accomplishing the above objects and of fulfilling the needs above mentioned and finally of providing the advantages above mentioned, will become apparent from the detailed description of the invention which follows.

### BRIEF SUMMARY OF THE INVENTION

A tourniquet dressing sleeve for digits is made from a sleeve of fabrifoam polymeric stretchable material layered with a covering layer of stretchable woven fabric, then folded over upon itself to form a sleeve with the edges sewn together to form a flexible, stretchable compression sleeve to fit around digits to exsanguinate them.

### BRIEF DESCRIPTION OF THE DRAWINGS

FIG. 1 is a perspective view of the exsanguinating tourniquet sleeve of the present invention.

FIG. 2 is an end view of the unit of FIG. 1 along 2-2 of FIG. 1.

FIG. 3 is a sectional view along line 3-3 of FIG. 2.

### DETAILED DESCRIPTION OF THE PREFERRED EMBODIMENT

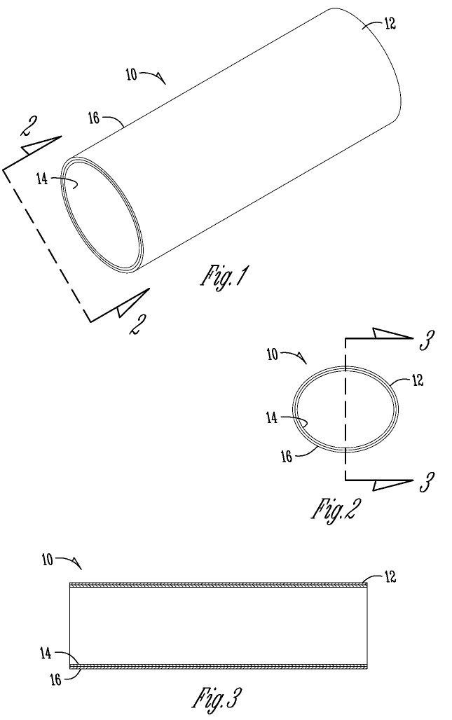

Exsanguinating tourniquet 10 shown in FIG. 1 comprises an elongated tube 12 made from an inner tubular material of fabric like polymer foam 14 and an exterior woven material stretchable surface 16. The foam material may be a natural rubber, synthetic rubber, and an open or closed cell foam material. It can be made from materials such as urethane, polypropane, ethylene, etc. Basically it will be a white foam material because of its contact with the human skin, and typically sterile. One suitable material is sold under the trademark “Fabrifoam”, and is available from Fabrifoam, Inc. of Exton, Pa. 19341.

Fabrifoam is a patented composite material combining an open celled, elastomeric, non-latex foam with selected high quality and specifically engineered fabrics. It is used by medical professionals including: physicians, physical therapists and certified athletic trainers to provide compression and support therapy to treat their patients soft tissue injuries. It is breathable; resists migration; optimizes hot/cold therapy; wicks away perspiration; provides maximum compression; infinitely adjustable; and is safe, washable and highly durable.

Surrounding the preferred non-latex polymer 12 is a stretchable material in order to have the stretch and pull characteristics of for example nylon hose. It can be natural or synthetic as a woven polymeric material. The two materials are adhered together adhesively. Sheets of the two adhered materials are thereafter folded over upon themselves to form a seam line which is sewn together to form the sleeve. Sleeves may be cut to individual lengths for various digits for all sizes of patients. They are then packaged as sterilized and individually useable and sold in boxes, for example of 27 units with three different lengths and diameters. The benefits of use of this device include: modest stretch in the material making for easy application over the digit or toes; when rolled on the material tightens around the digit or toe causing exsanguination; and when unrolled on the digit the material provides an attractive compression wrap for the patient. Typically the exterior woven polymer like material will be of a different color than skin so it can be easily discerned and thus avoid the possible accident of putting dressing materials on top of it, etc.

It therefore can be seen that the tourniquet dressing sleeve 10 is made from the fabrifoam 12, sewn and formed to a tube configuration with both ends open. When used as an exsanguinating tourniquet for digit surgical procedures it acts as a compressive dressing following the procedure to help with the hemostasis as well as provide with a smaller more uniform clean dressing. It has been developed to target health care providers who normally use a digit tourniquet when performing surgical procedures such as removing foreign bodies, removing ingrown toenails or soft tissue masses, nevus or warts from either toes or fingers. The advantages are several: it eliminates the need for hemostat and rubber band or the need for a Penrose drain; it allows for exsanguination of the digit; it reduces the need for extra dressing materials; it shortens the time for ingrown nail procedures; it affords more uniform, consistent compression as a tourniquet; it decreases the chance for contamination of a Coban roll or gauze roll; the learning curve is small; it is less likely to inadvertently be left on and then apply dressing over it; and the appearance is a cleaner more professional, consistent dressing.

It therefore can be seen that the invention accomplishes at least all its stated objections.

What is claimed is:

1. A tourniquet dressing sleeve for digits, comprising:
a sleeve of fabrifoam polymeric stretchable material layered with a covering of stretchable woven fabric and cut to a predetermined length to fit over and around human digits to exsanguinate them and compress the digit.

2. The tourniquet dressing sleeve of claim 1 wherein the sleeve is formed from a sheet of fabrifoam, layered with a sheet of stretchable woven fabric, folded upon themselves to form an elongated sleeve having an elongated edge, said edge being sewn to form a closed sleeve.

3. The tourniquet dressing sleeve of claim 2 wherein the stretchable woven fabric is a color that contrasts with skin color.

4. A method of exsanguinating digits, comprising
applying to and rolling on the digit to stop the blood from flowing a sleeve of fabrifoam polymeric stretchable material layered with a covering of stretchable woven fabric and cut to a predetermined length to fit over and around human digits to exsanguinate them and compress the digit.
